(19) United States
(12) Patent Application Publication
(10) Pub. No.: US 2017/0315203 A1
(43) Pub. Date: Nov. 2, 2017

(54) PERFORATOR PHASE CONTRAST ANGIOGRAPHY (pPCA)

(71) Applicant: Ohio State Innovation Foundation, Columbus, OH (US)

(72) Inventors: Michael V. Knopp, Columbus, OH (US); Xiangyu Yang, Columbus, OH (US); Michael J. Miller, Columbus, OH (US)

(21) Appl. No.: 15/141,927

(22) Filed: Apr. 29, 2016

(51) Int. Cl. G01R 33/563; A61B 5/026; G06T 5/50; A61B 5/0285

(52) U.S. Cl. CPC G01R 33/5635; A61B 5/0285; G01R 33/56316; G06T 5/50; A61B 5/026; G06T 2207/10088; G06T 2207/20221; G06T 2207/30104; G06T 2200/04

(57) ABSTRACT

The present disclosure is directed to methods and systems for fusing Phase Contrast Angiography (PCA) with anatomic images to create a perforator PCA (pPCA) data set. In the pPCA) method, vascular and anatomic information may be provided by different MRI sequences. A four-point acquisition scheme may be used for 3D PCA acquisition of vascular images. Anatomical MRI images are acquired and may be enhanced with image post-processing techniques. The vascular and anatomical images may be combined with image fusion to create a high resolution map of abdominal wall vasculature. This high resolution map visualizes not only the size and location of the DIEP perforators, but also their relationship with surrounding tissue, and the blood flow velocity within them. As such, the fused pPCA image has substantially higher SNR and CNR than CTA image of the same slice thickness.

### BACKGROUND OF THE DISCLOSURE

Surgical flaps are units of tissue defined by a specific blood supply that allow rearrangement or transfer of the tissue from one area of the body to another in order to restore functional and aesthetic deformities. It is a widely used method in plastic surgery to restore large defects caused by missing or damaged tissues associated with trauma, tumor resection, congenital malformation, or degenerative processes. Establishing and maintaining sufficient blood supply to the transferred tissue is crucial for uncomplicated healing and a successful outcome. The most advanced tissue transfer techniques utilize flaps based upon single blood vessels that pass through muscles and fascia into the subcutaneous fat called perforating vessel. In these so-called perforator flap surgeries, the appropriate choice of perforator is the key to success. Suitable perforators must be located in an area that allows safe surgical dissection, large enough to allow microsurgical repair (i.e., generally >1 mm in diameter), and adequate to supply all portions of the transferred tissues.

It is an increasingly common practice in plastic surgery to plan perforator flap procedures using non-invasive imaging technologies to map the vascular anatomy of the flap preoperatively to confirm perforator location, size, and architecture. Doppler Ultrasound (US) and Computed Tomographic Angiography (CTA) have been the most widely used preoperative imaging techniques for planning perforator flap surgery. Doppler US is inexpensive and widely available, but the quality of information yielded is highly dependent on operator skill. For this reason, CTA has gradually replaced Doppler US and become the gold-standard for perforator imaging. Despite higher cost, CTA is preferred clinically because it provides 3D visualization that is more robust, accurate, and intuitive for the operating surgeon. Reported benefits of CTA include shorter surgery time, reduced emotional stress for the surgeon, and reduced postoperative complications. Disadvantages of CTA include lack of information regarding flow velocity and venous anatomy as well as increased patient risk associated with exposure to ionizing radiation and iodinated contrast media.

A number of alternative perforator imaging methods using Contrast-Enhanced Magnetic Resonance Angiography (CE-MRA) techniques have been proposed. These methods are essentially analogy to the CTA method, with the iodinated CT contrast media replaced by Gadolinium-based MR contrast media. Therefore they also share the weakness on the lack of velocity and venous information with CTA. The safety profile of CE-MRA is generally better than the CTA method due to the use of non-ionizing radio-frequency (RF) radiation in MRI and the lower adverse reaction rates of the Gadolinium-based contrast media, but there is still a risk of causing nephrogenic systemic fibrosis (NSF) in patients with severely impaired renal function. Moreover, the safety advantage of CE-MRA is largely counterweighted by its lower spatial resolution and poorer vessel contrast than CTA. Consequently, none of the existing CE-MRA methods has found main-stream application in the imaging of perforators.

Non-contrast MRA methods, such as Time-Of-Flight (TOF) angiography and Phase Contrast Angiography (PCA), have been introduced as early as in the 1980s. These non-contrast MRA techniques enhance vessel contrast through suppressing signals from stationary tissue. This lack of stationary tissue contrast (i.e. fat/muscle contrast) is a major drawback for the application of perforator imaging, as the plastic surgeons has as much interest in a perforator's relationship with its environment as in the perforator itself. In addition, the spatial resolution of those non-contrast MRA techniques is limited by the signal-to-noise ratio (SNR), clinically acceptable scanning time, and the associated vulnerability to motion artifact, to a level that is typically worse than that of CE-MRA. Therefore, non-contrast MRA techniques are conventionally considered as being unsuitable for perforator imaging.

### SUMMARY OF THE DISCLOSURE

The present disclosure is directed to methods and systems for fusing PCA with anatomic images to create a perforator PCA (pPCA) data set. A pPCA protocol is described that is optimized for, e.g., deep inferior epigastric perforator (DIEP) imaging. DIEP flaps are widely used in autologous breast reconstruction after mastectomy. A study is presented that demonstrates the feasibility of in vivo perforator visualization with pPCA as an alternative to CTA, and to prospectively compare these two techniques in terms of overall image quality, delineation of perforator anatomy (i.e., diameter, location, and intramuscular course), and clinical value.

In accordance with the present disclosure, a method for Phase Contrast Angiography (pPCA) is provided. The method includes acquiring vascular and flow information using a first MRI sequence; acquiring anatomic information using a second MRI sequence; reversing a contrast of the anatomic information to create reversed anatomic information; and creating a high resolution map of vasculature from the reversed anatomic information and vascular and flow information.

In accordance with the present disclosure a magnetic resonance imaging (MRI) apparatus is disclosed that includes a magnet, gradient coils, radio frequency (RF) coils, and a controller. The controller is programmed to execute instructions to perform a method of Phase Contrast Angiography (pPCA) that includes acquiring vascular and flow information using a first MRI sequence; acquiring anatomic information using a second MRI sequence; reversing a contrast of the anatomic information to create reversed anatomic information; and creating a high resolution map of vasculature from the reversed anatomic information and vascular and flow information.

Other systems, methods, features and/or advantages will be or may become apparent to one with skill in the art upon examination of the following drawings and detailed description. It is intended that all such additional systems, methods, features and/or advantages be included within this description and be protected by the accompanying claims

### BRIEF DESCRIPTION OF THE FIGURES

FIG. 1 is an overview of the processes performed in accordance with the present disclosure;

FIGS. 2A-2B illustrate example imagery obtained from a volunteer patient;

FIGS. 3A-3E illustrate aspects of image quality and clinical value of pPCA and CTA;

FIG. 4A illustrates an axial pPCA image of a perforator on the left side of a patient;

FIG. 4B illustrates a CTA maximum intensity projection (˜100 mm thickness) of a perforator on the left side of the patient in FIG. 4A;

FIG. 5A shows a 1.5 mm thick axial pPCA image that resolves two parallel perforator vessels next to each other in the rectus abdominis muscle of a patient;

FIG. 5B shows a CTA image with the same slice thickness which shows only one perforator vessel is visible in the same patient

FIGS. 5C and 5D show in the axial thick-slice maximum intensity projection of CTA, the two parallel vessels that diverge in the subcutaneous fat layer and are blurred together and appear as a single perforator with subcutaneous bifurcation; and

FIGS. 6A-6B illustrate images showing coronal pPCA and CTA maximum intensity projection of the deep inferior epigastric arteries (DIEA) in a patient.

FIG. 7A shows that scanning the patient in prone position may causes craniocaudal and lateral deviations of the measured perforator location in pPCA;

FIG. 7B shows that there is a strong correlation between the craniocaudal deviation and patient BMI; and

FIG. 7C shows that no correlation is found between the lateral deviation and patient BMI.

### DETAILED DESCRIPTION OF THE DISCLOSURE

Described herein is a method of creating high resolution 3D maps of perforator vessels and their surrounding tissues with non-contrast MRI techniques, which overcomes the limitations of other existing methods by enabling simultaneous acquisition, visualization, and assessment of vessel, flow, and soft-tissue information. With the multi-dimensionality of the information provided by method, a computer-assisted surgical planning system can be created to facilitate decision making in various plastic surgeries using perforator flap techniques.

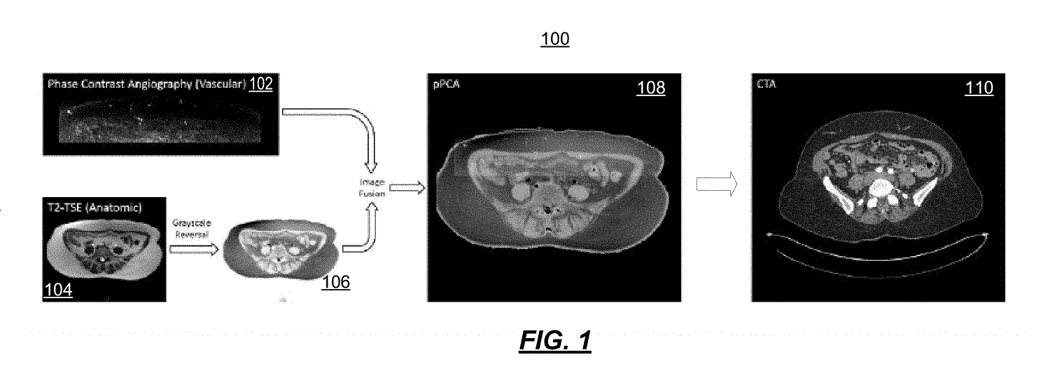

FIG. 1 is an overview of an example perforator Phase Contrast Angiography (pPCA) method 100 performed in accordance with the present disclosure. Phase Contrast Angiography (PCA) is a non-contrast Magnetic Resonance Angiography (MRA) technique that uses bipolar gradients to encode tissue movement with velocity-dependent phase shift. PCA has favorable features that potentially overcome the limitations of other techniques, such as absence of ionizing radiation and contrast media, sensitivity to flow velocity, and full 3D visualization capability. In the perforator Phase Contrast Angiography (pPCA) method, the vascular (102) and anatomic information (104) may be provided by different MRI sequences.

MR signals may be acquired at 102 with a phased-array receiver coil constructed of a large number of small coil elements. It is known that a RF coil's SNR and penetration depth are governed by the dimension of its individual coil elements. A receiver coil designed in this way can yield high SNR in a layer of subcutaneous tissue near the coil surface, in which the perforator vessels of interest are located, and enable visualization of perforator vessels with submillimeter resolution in clinically acceptable acquisition time. The limited penetration depth of such a coil helps to suppress MR signals from deeper tissues, which are irrelevant for the perforator flap surgery and often become sources of motion artifacts. Motion control techniques, such as respiratory triggering and/or saturation bands, can be applied when imaging body parts that are especially prone to physiologic and/or voluntary motions, such as the abdominal or thoracic regions.

The coil may have a flexible design so that it can be positioned in close proximity to the body part of interest. Ergonomically-designed positioning accessories may be added to improve patient comfort and/or provide physical restrictions to minimize patient motion. The vascular and flow information is then acquired with a 3D phase contrast technique, using a maximum encoded velocity substantially lower than typically used in arterial or venous PCA applications (e.g., for the Deep Inferior Epigastric Perforators, a maximum encoded velocity of ˜15 cm/s may be used). The high SNR of the coil described above allows the PCA data to be acquired with sufficiently high in-plane resolution comparable to that of the state-of-the-art multi-detector CTs (for example, 0.5×0.5 mm in-plane resolution and 1.5 mm reconstructed slice thickness can be achieved with a 350×100 mm field of view over a scan length of 200 mm within a 10 to 15 minutes acquisition).

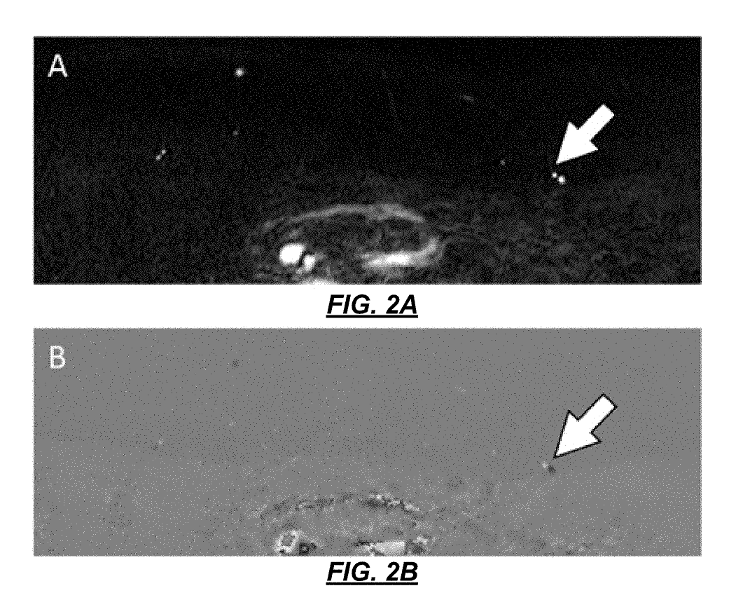

In accordance with the present disclosure, a four-point acquisition scheme may be used for the 3D PCA acquisition so that all three orthogonal components of the flow velocity vector can be obtained. The amplitude of the flow velocity vector can be used for perforator visualization and velocity measurement. The direction of the flow velocity vector can be used for the differentiation of arterial and venous flows when combined with the a priori knowledge of blood flow patterns in the body part imaged, as shown in FIGS. 2A-2B. In FIGS. 2A-2B, there are shown examples from a 36-year old male volunteer demonstrates that perforator artery vein in a parallel pair (see, the two bright dots marked out by the arrow in subplot A) can be differentiated with the opposite directions of blood flow along the head-foot direction (subplot B, with the white color denotes a positive velocity component and the black color denotes a negative component).

The morphological MRI images can be acquired at 104 with any MRI sequence, provided that it can generate images with good soft tissue contrast. The spatial coverage and resolution of the morphological images need to be comparable to that of the 3D PCA acquisition. The morphological images can be further enhanced with image post-processing techniques (For example, the contrast of standard T2-weighted Turbo Spin Echo images can be inverted through post-processing to create CTA-like soft-tissue contrast). No external marker or internal landmark is needed for such co-registration.

At 106, in order to obtain a soft tissue contrast comparable to that of the CTA, the contrast of the anatomic T2-TSE data acquired at 104 is reversed.

At 108, the images data from 102 and 106 are combined with image fusion to create a high resolution map of abdominal wall vasculature. In particular, the flow vector field obtained with the 3D PCA acquisition at 102 is combined with the anatomical information provided by one or more morphological MRI acquisitions at 106 through, e.g., image co-registration. This high resolution map visualizes not only the size and location of the DIEP perforators, but also their relationship with surrounding tissue, and the blood flow velocity within them. The product of the flow velocity within a perforator vessel and its diameter can provide the plastic surgeons a metric of this perforator vessel's perfusion capability, which could be valuable for the optimization of surgical plans and the prevention of post-surgical complications. As such, the fused pPCA image generated at 108 has substantially higher SNR and CNR than CTA image of the same slice thickness (see example at 110).

The co-registered data set obtained with the method described above (i.e., perforator phase contrast angiography, pPCA) has advantages over the existing perforator imaging methods, as listed in Table 1 below:

With all the vascular, anatomical, and flow information obtained as one package with the pPCA method 100 described above, a computer-assisted surgical planning system may facilitate automatic or semi-automatic extraction of the crucial information needed by the plastic surgeon to perform a perforator flap surgery. Such a system may include, but are not limited to, automatic arterial/venous flow identification, automatic perforator detection, tracking of perforator intramuscular course, perforator diameter measurement, and perforator perfusion capability quantification. The system may also have sufficient 3D visualization capabilities to make this information conveniently accessible to the surgeons. The system can be integrated into the pre-operative decision chain to improve the efficiency of surgical planning, reduce the inter-operator variability associated with non-standardized utilization of imaging data, and eventually improve surgical outcomes.

In accordance with aspects of the disclosure, such a computer-aided surgical planning system may be implemented as a cloud-based service, independent of the computational resources available to the plastic surgeons, to which the image data shall be uploaded together with relevant clinical information, and subsequently the completely processed data will be made available online to the plastic surgeons.

If the pPCA method 100 is applied to both the donor-site and the site of defects to be repaired, the system could also be used for a “virtual surgery” application. Such an application may be useful in difficulty cases and/or for training/educational purposes. The computer-assisted surgical planning system may be further developed and integrated into an intraoperative image guidance system.

Another application of the pPCA method 100 and computer-assisted surgical planning system is to provide personalized flap design. The pPCA method and the computer-assisted surgical planning system are generally applicable to all major types of perforator flap surgery, including but are not limited to Deep Inferior Epigastric Perforator (DIEP) flap, Anterolateral Thigh (ALT) flap, Transverse Rectus Abdominis Myocutaneous (TRAM) flap, Superficial Inferior Epigastric Artery (SIEA) flap, Superior Gluteal Artery Perforator (SGAP) flap, and Inferior Gluteal Artery Perforator (IGAP) flap.

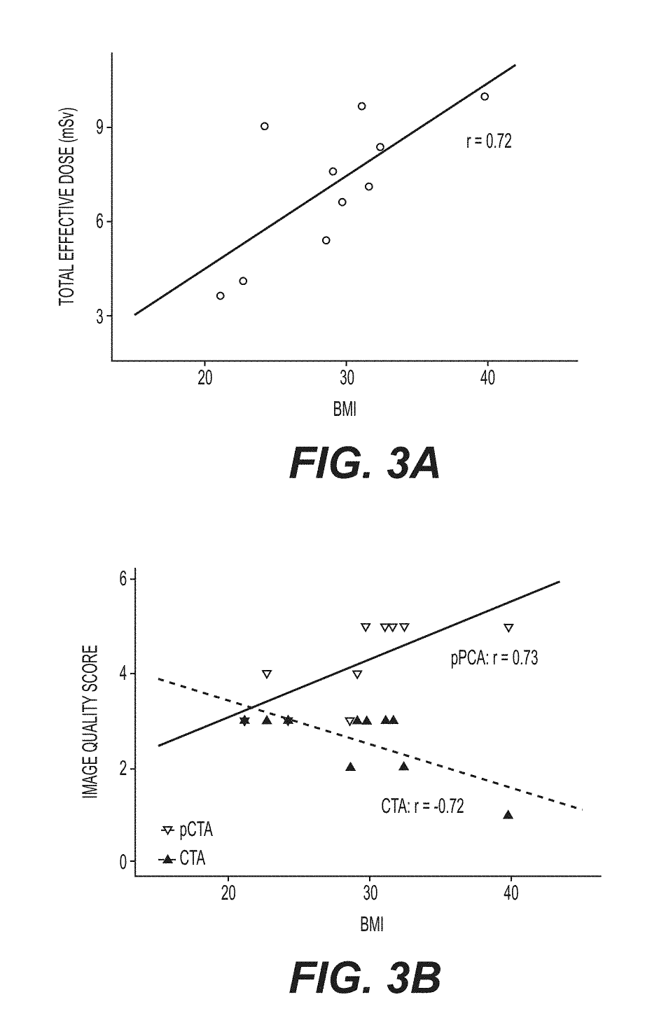

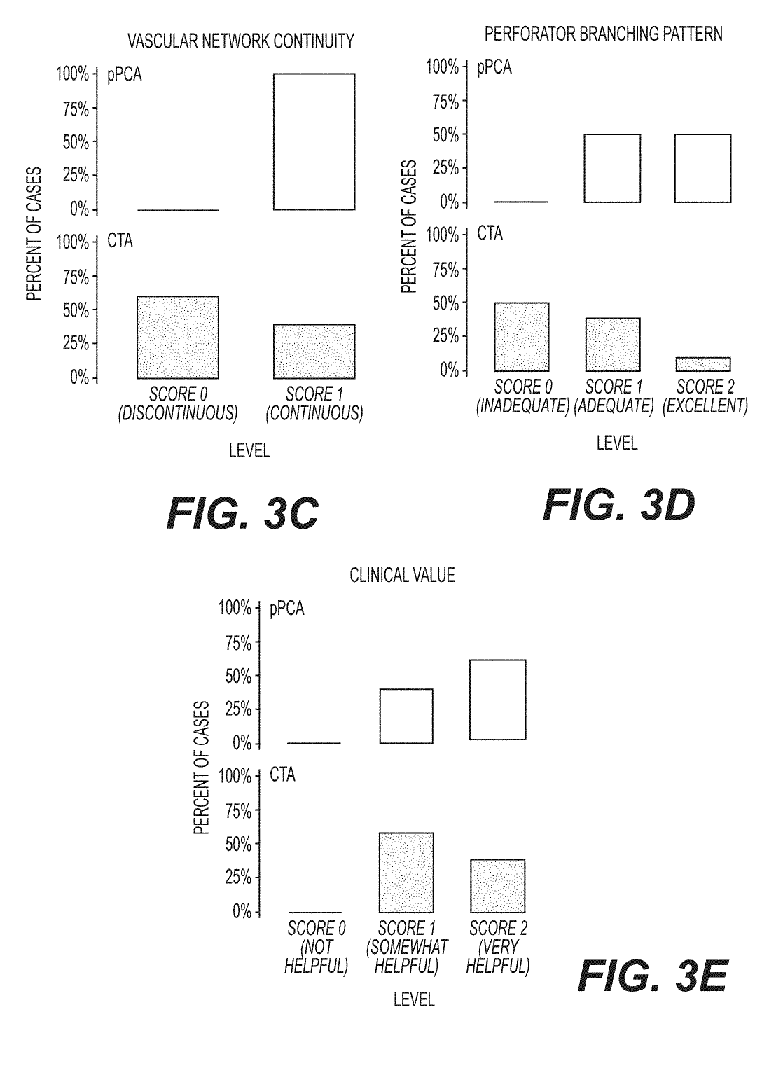

FIGS. 3A-3E illustrate aspects of image quality and clinical value of pPCA and CTA. FIG. 3A shows that the dose of DIEP CTA is positively correlated with patient body mass index (BMI). FIG. 3B shows that image quality of pPCA is positively correlated with patient BMI. Image quality of CTA is negatively correlated with patient BMI. This observation suggests that obese and overweight patients (with BMI≧25.0) will get the most benefit from the pPCA technique. FIG. 3C shows that as compared with pPCA, a significantly higher percentage of CTA cases has discontinuity in the visualized vascular network. FIG. 3D shows that pPCA also has less cases of “inadequate” visualization of perforator branching patterns and more cases of “excellent” visualization. FIG. 3E shows that more pPCA cases are rated as being “very helpful” by the Plastic Surgeon than DIEP CTA (although the different is not statistically significant).

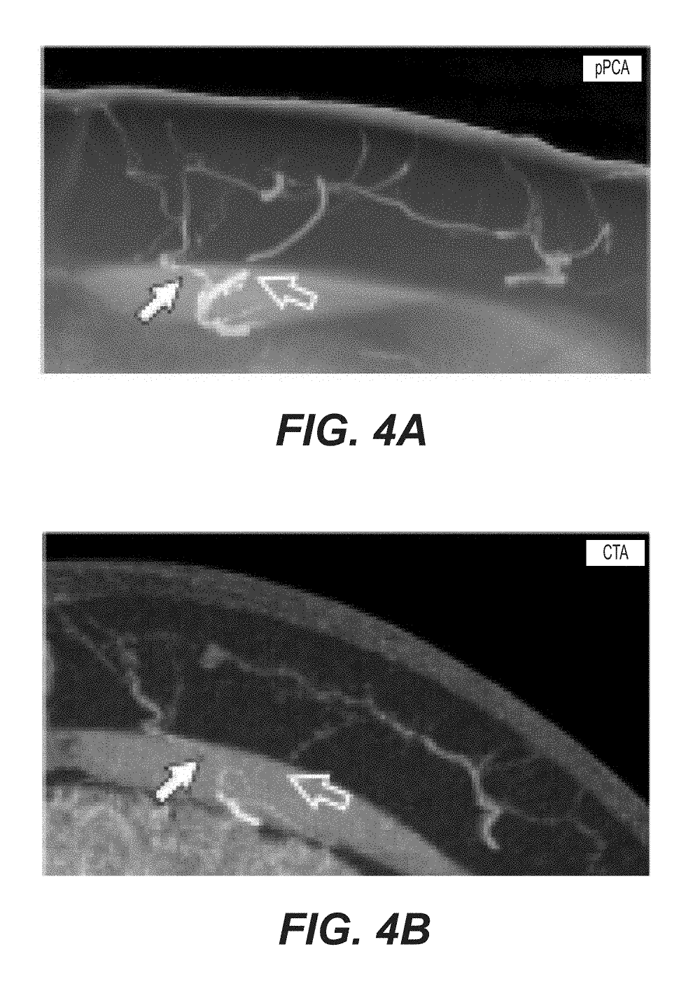

FIG. 4A illustrates an axial pPCA image of a perforator on the left side of a patient. FIG. 4B illustrates a CTA maximum intensity projection (˜100 mm thickness) of a perforator on the left side of the patient in FIG. 4A. High SNR and CNR of pPCA allows better visualization of this perforator in both the rectus abdominis muscle and the subcutaneous fat. In this patient, poor visualization of this perforator's intramuscular section by CTA (white solid arrow) even led to erroneous determination of its direction: the reviewer had difficulty in precisely identifying the locations of the entry and the exit points of this perforator on CTA. Eventually, an inconclusive assessment was made based on an assumption that the exit point is probably cranial to the entry point. However, the pPCA data clearly demonstrate that the exit point is on the caudal side of the entry point. A second branch of this perforator (white hollow arrow) is also completely missed by CTA.

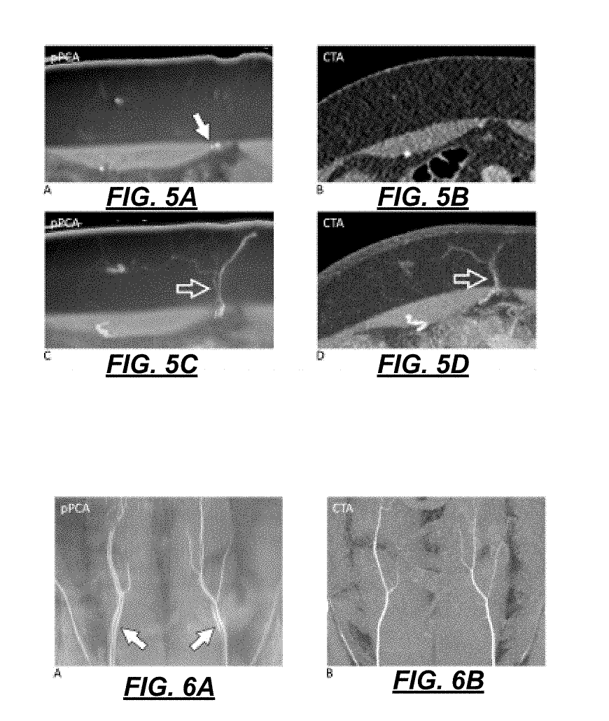

FIG. 5A shows a 1.5 mm thick axial pPCA image that resolves two parallel perforator vessels next to each other in the rectus abdominis muscle of a patient. FIG. 5B shows a CTA image with the same slice thickness which shows only one perforator vessel is visible in the same patient. FIGS. 5C and 5D show in the axial thick-slice (˜47 mm thickness) maximum intensity projection of CTA, the two parallel vessels that diverge in the subcutaneous fat layer and are blurred together and appear as a single perforator with subcutaneous bifurcation (white hollow arrows).

FIGS. 6A-6B illustrate images showing coronal pPCA and CTA maximum intensity projection (˜30 mm thickness) of the deep inferior epigastric arteries (DIEA) in a patient. The deep inferior epigastric veins are not visible in the CTA image because the imaging data were acquired in the arterial phase. The pPCA technique does not have this limitation. Both arterial and venous flows can be detected and visualized with a single scan (thick white solid arrows).

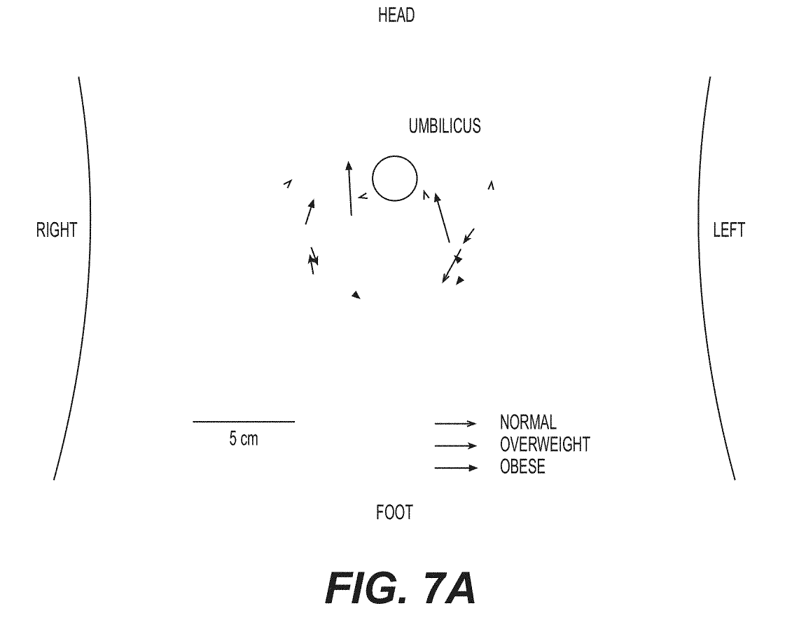

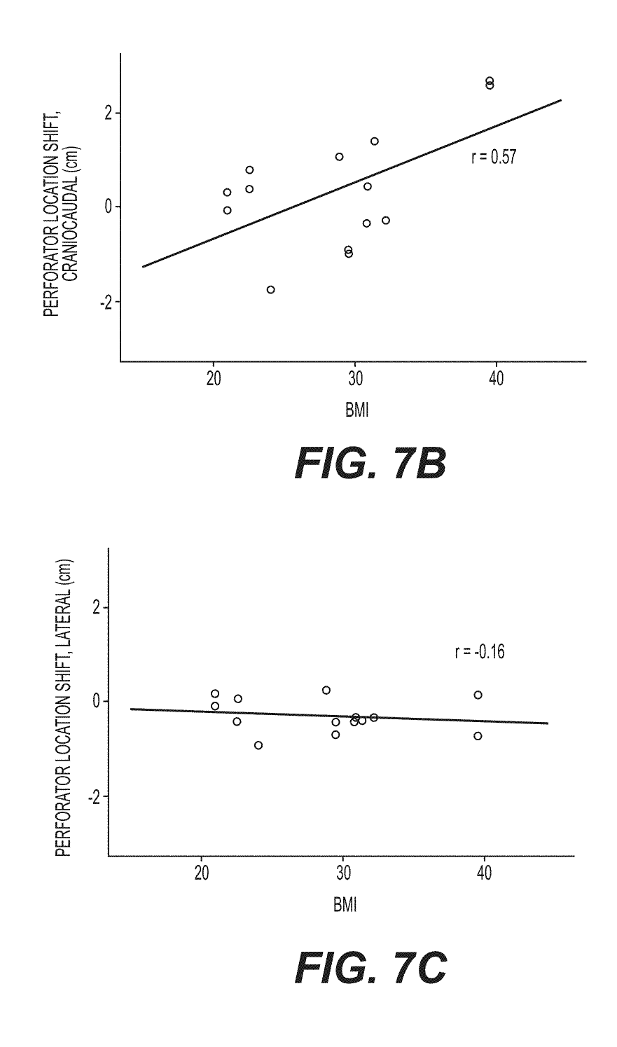

FIG. 7A shows that scanning the patient in prone position may causes craniocaudal and lateral deviations of the measured perforator location in pPCA. Each arrow represents the deviation of a POI's location between pPCA (the end point of the arrow) and CTA (the starting point of the arrow). The arrows are color-coded with the patient's body mass index (BMI), with blue representing normal patient (18.5≦BMI<25.0), orange representing overweight patient (25.0≦BMI<30.0), and red representing obese patient (BMI≧30.0). The size of the umbilicus is based on the data published by Yu et al. FIG. 7B shows that there is a strong correlation between the craniocaudal deviation and patient BMI. There is a strong correlation between the craniocaudal deviation and patient BMI (a positive number represents a cranial deviation in pPCA), indicating that larger patients tend to have more deviation along this direction. FIG. 7C shows that no correlation is found between the lateral deviation and patient BMI. No correlation is found between the lateral deviation and patient BMI (a positive number represents a distal deviation in pPCA).

Study

Patient Population

Ten patients were recruited from a population of females undergoing DIEP flap breast reconstruction after total mastectomy for breast cancer treatment. Exclusion criteria include: contraindication to iodinated contrast media, activatable implants or metallic foreign objects in body, permanent tattoos, and claustrophobia. The average age of the patient population is 57 years (range: 42-69 years). The average body mass index (BMI) is 29.0 (range: 21.1-39.8). Two male volunteers (33/26 years old, BMI 26.2/26.8) were also recruited for the optimization of the DIEP pPCA protocol.

Imaging

The DIEP pPCA protocol of the present disclosure was optimized with volunteers on a 3T MRI (Achieva, Philips Healthcare, Cleveland, Ohio, USA) prior to patient studies. The MRI apparatus was programmed to perform the DIEP pPCA as disclosed herein. The subjects were scanned in prone position with a 32-channel cardiac coil (Philips Healthcare, Cleveland, Ohio, USA). Parallel transmit technology was used to improve B₁ homogeneity over large field-of-view (14). The optimized pPCA protocol contains a high resolution axial PCA sequence and an axial T2-weighted Turbo Spin-Echo (TSE) anatomic sequence (Table 2). Both sequences are respiratory triggered. Depending on the subject's respiration rate, it takes approximately 15-25 minutes to acquire the entire protocol.

TABLE 2
Sequence Parameters of the DIEP pPCA Protocol

| | | PCA | T2-TSE |
|---|---|---|---|
| Sequence Parameters | | (Vascular) | (Anatomic) |
| Field-of-View | RL | 350 | 375-440 |
| (mm) | AP | 100 | 300 |
| | FH | 200 | 198 |
| Resolution | In-plane | 0.5 | 0.7 |
| (mm) | Slice | 1.5 | 5 |
| | Gap | 0 | 0.5 |
| TR (ms) | | 8.7 | 801-838 |
| TE (ms) | | 5.2 | 80 |
| Flip Angle (°) | Excitation | 11 | 90 |
| | Refocusing | N/A | 120 |

| | | |
|---|---|---|
| Bandwidth (Hz) | 283.4 | 532.8-537.5 |
| Motion Compensation | Respiratory Trigger | Respiratory Trigger |
| Saturation Band | Posterior | None |
| Others | Vₘₐₓ = 15 cm/s; | N/A |
| | Only uses anterior coil. | |

All study patients underwent preoperative DIEP pPCA using the optimized protocol and DIEP CTA. The CTA study was performed in supine position on a multi-detector CT (Somatom Definition AS+, Siemens Healthcare, Erlangen, Germany) with intravenous injection of an iodinated contrast medium (Omnipaque, GE Healthcare, Princeton, N.J., USA; weight-based dose) at an injection rate of 4 ml/sec. Contrast arrival was monitored by a bolus tracking Region-Of-Interest placed in femoral artery. The CTA data were acquired at 100 kVp (except for one patient, for whom 120 kVp was used), and reconstructed to 0.75 mm slice thickness using iterative reconstruction. Dose modulation was used in all studies.

Data Analysis

All imaging data were evaluated on an Intellispace Portal workstation (Philips Healthcare, Cleveland, Ohio, USA). Both the pPCA and CTA data were reviewed in transverse plane at identical slice thickness (1.5 mm) and magnification. For the pPCA data, the vascular and anatomic information in different MRI sequences were combined by fusing the PCA images with grayscale-inversed T2-TSE images. For the CTA data, thicker (up to 30 mm) maximum-intensity projections (MIP) were occasionally used for perforator identification when the signal-to-noise ratio (SNR) of the 1.5 mm MIP was low.

Clinical assessment of the imaging data was performed by a senior Plastic Surgeon (with 25 years of experience in breast reconstruction using microvascular techniques) in a manner similar to that used in routine clinical practice. After anonymization and randomization, each imaging data set was evaluated independently and blindly, with a separation in time of at least three months between the analysis of pPCA and CTA data to minimize potential recall bias. On each side of the abdomen, the reviewer identified significant perforating vessels, and selected one as the perforator-of-interest (POI), which appeared to be the most favorable for potential microvascular tissue transfer based on the reviewer's clinical judgement. For each POI, the perforator's location, diameter, SNR, contrast-to-noise ratio (CNR), and the length of its intramuscular course was measured. The overall image quality and qualitative assessment of clinical value was rated with a set of categorical scores (Table 3).

TABLE 3
Image Assessment Metrics
Categorical Scores

| | |
|---|---|
| Image Quality | Levels: |
| | 0 (lowest) to 5 (highest). |
| Vascular Network Continuity * | Levels: |
| | 0-Discontinuous; |
| | 1-Continuous. |
| Clinical Value | Levels: |
| | 0-No helpful information; |
| | 1-Somewhat helpful, with concerns; |
| | 2-Very helpful, support critical decisions. |
| Perforator Branching Patterns | Levels: |
| in the Subcutaneous Tissue ^{†} | 0-Inadequate visualization; |
| | 1-Adequate visualization; |
| | 2-Excellent visualization. |

Quantitative Metrics

| | |
|---|---|
| Signal to Noise Ratio (SNR) | SNR = Perforator Signal/Noise ^{†} |
| | (Dimensionless Quantity) |
| Contrast to Noise Ratio, | CNRₘ = (Perforator Signal-Muscle Signal)/Noise ^{‡} |
| Perforator-to-Muscle (CNRₘ) | (Dimensionless Quantity) |
| Contrast to Noise Ratio, | CNR_{f} = (Perforator Signal-Fat Signal)/Noise ^{‡} |
| Perforator-to-Fat (CNR_{f}) | (Dimensionless Quantity) |
| Perforator Location | The lateral and craniocaudal distances between the |
| | perforator's exit point from the rectus sheath and |
| | a fixed anatomic landmark (the lower border of |
| | the umbilicus; in cm). |
| Perforator Diameter | Measured at the perforator's exit point from the |
| | rectus sheath (in mm). |
| Intramuscular Course | The straight-line distance between the perforator's |
| | entry point to and exit point from the rectus |
| | sheath (in cm). |

* Continuity was defined as a visible continuous course from the origin of the deep inferior epigastric vessels on the common femoral vessels, through the rectus abdominis muscle and anterior rectus fascial sheath, and into the subcutaneous tissues.
^{†} The visualization of the vascular branching pattern in the subcutaneous space is an indicator of the adequacy of perfusion of different regions of the flap.
^{‡} The noise was measured as the standard deviation within a fat Region-Of-Interest with an area of approximately 150 mm² and no obvious internal structures.

Statistical analysis was performed using the R language (The R Foundation, Vienna, Austria). All continuous measurements were compared with paired student's t-test after the normality was confirmed by the Shapiro-Wilk normality test and the Q-Q plot. The 6-level image quality score was also treated as continuous and compared using t-test. All other categorical scores were compared using Fisher's exact test. All P values were adjusted with the Benjamini-Hochberg false discovery rate (FDR) method (controlling FDR at 0.05) for multi-comparison correction.

Results

All imaging studies were successfully performed. The average total effective dose of CTA is 7.14 mSv (range: 3.63-9.98 mSv) in this patient cohort. There is a positive correlation between dose and patient BMI (r=0.72; FIG. 3A).

Image assessment results and the corresponding statistics are summarized in Table 4.

TABLE 4
Summary of Image Assessment Results.

| | | pPCA | CTA | FDR-Adjusted P |
|---|---|---|---|---|
| Categorical | Vascular Network Continuity | | | |
| Scores | 0: Discountinuous | 0% | 60% | 0.014*^{†} |
| (N = 10) | 1: Continuous | 100% | 40% | — |
| | Clinical Value | | | |
| | 0: Not Helpful | 0% | 0% | 1.000^{†} |
| | 1: Somewhat Helpful | 40% | 60% | — |
| | 2: Very Helpful | 60% | 40% | 0.388^{†} |
| | Perforator Branching Patterns | | | |
| | 0: Inadequate | 0% | 50% | 0.026*^{†} |
| | 1: Adequate | 50% | 40% | — |
| | 2: Excellent | 50% | 10% | 0.102^{†} |
| | Image Quality | | | |
| | 0 (Lowest) to 6 (Highest) | 4.2 ± 0.9 | 2.6 ± 0.7 | 0.005*^{†} |
| Quantitative | SNR | 31.4 ± 16.0 | 9.4 ± 4.8 | <0.001*^{‡} |
| Metrics | CNR | | | |
| (N = 14) | Perforator-to-Muscle | 26.3 ± 15.9 | 5.5 ± 4.6 | <0.001*^{‡} |
| | Perforator-to-Fat | 29.2 ± 16.0 | 16.2 ± 5.7 | 0.014*^{†} |
| | Perforator Location (cm) | | | |
| | Lateral | 3.0 ± 1.2 | 3.3 ± 1.2 | 0.021*^{‡} |
| | Craniocaudal | 2.4 ± 2.3 | 2.8 ± 1.7 | 0.359^{‡} |
| | Perforator Diameter (mm) | 2.0 ± 1.1 | 2.7 ± 1.2 | <0.001*^{‡} |
| | Intramuscular Course (cm)^{§} | 3.6 ± 2.5 | 3.9 ± 2.0 | 0.415^{‡} |

*Statistically significant findings.
^{†}Fisher's exact test
^{‡}Paired Student's t-test
^{§}N = 11

Image Quality and Clinical Value

Image quality is found to be negatively correlated with patient BMI for DIEP CTA (r=−0.72), but positively correlated with patient BMI for the MR-based pPCA (r=0.73; FIG. 3B). Overall, pPCA has significantly higher image quality than CTA (P=0.005). The two imaging modalities are only considered equivalent in two patients with low BMI.

Discontinuity in visualized vascular network was noticed in 60% of the CTA cases, but not in any of the pPCA data set (P=0.014; FIG. 3C). The reviewer rated visualization of perforator branching patterns in the subcutaneous tissue as “inadequate” in 50% of the CTA cases, while all pPCA cases were rated as “adequate” or “excellent” (P=0.026; FIG. 3D).

As for the clinical value of the imaging data, all imaging studies were found to provide clinically helpful information that supports surgical decisions-making at some level. A higher percentage of pPCA cases are rated as being “very helpful” (pPCA: 60%, CTA: 40%; FIG. 3E), but the difference does not reach statistical significance (P=0.388) in this small cohort.

Perforator Identification

Over the entire patient population, 81 perforators were identified on at least one angiogram. One was outside the MRI field-of-view and was thus excluded from analysis. Among the other 80 perforators, 84% ( 67/80) are observed on both pPCA and CTA. 9% ( 7/80) are only visible on pPCA. The other 7% ( 6/80) are only seen on CTA. The majority of the 6 perforators not seen on pPCA (BMI 21.1/24.2/31.1:4/1/1) are from two small patients with the lowest pPCA image quality score. Review of these data suggest that low perforator detectability is associated with motion artifacts in PCA images. Contrarily, the 7 perforators missed by CTA are more evenly distributed among all patient sizes (BMI 24.2/28.6/32.4/39.8:2/2/2/1), and are not associated with a particular type of imaging artifact.

In 70% ( 14/20) of cases, the reviewer chose the same perforator as POI on both pPCA and CTA, despite the fact that POI selection were independently and blindly made using the two different imaging modalities. Closer inspection of the remaining cases revealed the following reasons for the discrepancy in POI identification: lack of suitable perforator on that side (n=2), poor visualization of perforator intramuscular course by CTA (n=2), perforator location deviation between pPCA and CTA (n=1), and failure to visualize parallel vessels by CTA (n=1).

SNR and CNR

Measurements from the 14 POIs that have been selected on both angiograms demonstrate that PCA has significantly higher SNR (P<0.001), perforator-to-muscle CNR (P<0.001), and perforator-to-fat CNR (P=0.014) than CTA. Consequently, small perforators are better visualized with pPCA in both the rectus abdominis muscle and the subcutaneous fat. As demonstrated in the FIGS., imaging perforators with pPCA helps the Plastic Surgeon to reduce uncertainty in intramuscular course assessment (FIG. 4), to resolve nearby parallel vessels that could be blurred together by CTA (FIG. 5), and to detect both arterial and venous flows (FIG. 6).

Perforator Anatomy

In DIEP flap surgery, perforators are usually localized with a virtual grid system centered at the umbilicus. The DIEP pPCA study is performed with the patient in prone position to restrict respiratory motion of the abdominal wall. Tissue shift under pressure and rectus abdominis muscle stretch in prone position, however, may cause deviations in perforator location measurements. In order to assess the magnitude of this change, the lateral and craniocaudal coordinates of the 14 POIs measured by pPCA and CTA were compared.

In the lateral direction, scanning the patient in prone position causes a small (0.3±0.4 cm) but statistically significant (P=0.021) proximal shift of perforator coordinate in pPCA. In the craniocaudal direction, deviations can be substantially larger, but without a dominant shift direction (FIG. 7A). On average, perforators' craniocaudal coordinates of pPCA are 0.4±1.3 cm cranial to their locations in CTA. Deviations along this direction are statistically nonsignificant (P=0.359). There is a strong correlation between the craniocaudal deviation and patient BMI (r=0.57; FIG. 7B), but the correlation between the lateral deviation and patient BMI was negligible (r=−0.16; FIG. 7C).

On average, perforator size measured by pPCA is 0.8±0.3 mm smaller than the corresponding measurement from CTA, a statistically significant difference (P<0.001).

Among the 14 POIs, intramuscular course measurements were uncertain or inaccurate in two CTA cases (due to poor intramuscular course visualization) and one pPCA case (probably due to phase wrapping). In the other 11 POIs, the difference in intramuscular course assessment between the two imaging modalities was small (0.2±0.8 cm) and statistically nonsignificant (P=0.415).

Discussions

The data clearly demonstrates that a significant improvement in perforator imaging can be achieved with the MR-based pPCA technique described in this paper. Although the DIEP flap is provided as a example, all the MRI sequences utilized are widely available across all major manufacturers, and can be readily modified for utilization in other types of perforator flap surgery.

A major advantage of MRI as a preoperative imaging modality is the elimination of ionizing radiation and the associated risks. This is especially valuable for overweight (25.0≦BMI<30.0) and obese (BMI≧30.0) patients. With CT, these patients receive higher radiation doses than normal-sized patients (15), and their CT images usually have lower quality as a result of photon starvation. An important finding of this study is that the image quality of the MR-based pPCA technique improves with patient size, suggesting that these patients will get the most benefit from this new technique, in terms of both dose reduction and image quality improvement. Indeed, data reported in this paper already indicate that it is worthwhile to consider the MR-based pPCA as a preferable alternative to the X-ray-based CTA for preoperative DIEP imaging in overweight and obese patients.

Several perforator MRA techniques, mostly for DIEP imaging, have been proposed in literature (2, 16-20). Intravenous injection of Gadolinium-based contrast media is used by the majority of the existing techniques as a means to enhance perforators. The pPCA technique, conversely, does not require exogenous contrast medium. Eliminating the risk of adverse response to contrast agents (e.g., nephrogenic systemic fibrosis (21)) permits using this technique in patients with impaired renal function or allergy to contrast agents. In addition, non-contrast acquisition simplifies patient management and avoids interference with other contrast-enhanced imaging studies that might be required.

In recent years, DIEP contrast-enhanced MRA (CE-MRA) has undergone substantial improvement as a result of advances in imaging equipment, pulse sequences, and contrast media. However, it is still a consensus that CE-MRA has lower spatial resolution (19) and poorer contrast to fat (20) than CTA. The study demonstrates that pPCA overcomes those weaknesses. It has higher CNR than CTA in both muscle and fat. The 0.5 mm in-plane resolution is also comparable to the nominal axial resolution of advanced multi-detector CT. Moreover, because MRI reconstruction algorithms do not have an inherent blurring component, pPCA actually presents a noticeably better ability to resolve parallel perforator vessels.

Perforator Anatomy

In DIEP flap surgery, an optimal perforator should have a caliber large enough to perfuse the entire flap and exit the anterior rectus sheath somewhere between the umbilicus and the pubis. A direct course passing from the superficial to the deep surface of the muscle is also preferred for faster, safer, and less traumatic dissection. The ease and reliability of selecting the most suitable perforator on which to base the flap is contingent on this information, which is not possible for the surgeon to obtain intraoperatively before committing to a specific perforator to mobilize the flap. Consequently, it is highly advantageous to have it available from an accurate and reliable preoperative imaging modality.

Although an intraoperative validation in this imaging-focused methodology developmental study was not preformed, it is possible to obtain an indirect assessment for accuracy of the pPCA technique by comparing it to CTA, the well-established gold-standard for DIEP imaging. The study reveals a good agreement between the two techniques in perforator identification and intramuscular course measurement. Perforator size measurements are significantly smaller with the MR-based pPCA, which is probably more accurate according to studies published by Cina, et al., in 2010 and 2013. In these studies, intraoperative perforator caliber measurements were compared with imaging findings, and reported that CTA overestimates perforator size by 0.54±0.30 (22) to 1.18±0.35 mm (19), which is in excellent agreement with the observation that the pPCA perforator size measurements are 0.8±0.3 mm smaller than the CTA measurements. Interestingly, one of Cina's studies also reported CE-MRA is less accurate than CTA in perforator size measurement, with a mean error of 1.63±0.39 mm (19). The phase contrast technique of the present disclosure overcomes this weakness of CE-MRA, making it superior in this regard.

The deviations in perforator location observed in this study are larger than the reported perforator localization errors of CTA (0.5±0.2 cm) and CE-MRA (0.6±0.3 cm) (19), indicating that imaging patients in prone position impacts perforator localization accuracy, especially along the craniocaudal direction. Nevertheless, the pPCA technique appears adequate in practical terms for surgical planning, as the observed perforator location deviations are comparable to the precision of the virtual gird system (1 cm) and the size of the reference landmark (the umbilicus with its average size of 2.4±0.7 cm (23)), and it yields superior information on more critical aspects for perforator selection such as subcutaneous branching pattern and intramuscular course. Moreover, the study shows a strong correlation between craniocaudal deviation and patient BMI. This observation suggest a possibility of further improving pPCA's localization accuracy through more careful positioning of obese patients prior to MRI acquisition.

Limitations

The study is limited in its population size, as it was designed as a methodology developmental study. An intraoperative component and postoperative follow-up may be added to future prospective clinical trials based on this study, which will facilitate a direct assessment with clinical findings. The pPCA technique itself does have further potential for improvement, too. For example, there is no evidence that grayscale-inversed T2-TSE images are the best anatomic background for pPCA. Other imaging sequences, such as DIXON (24), may be able to provide more clinically relevant information with even higher resolution or shorter acquisition times. Similarly, development of image postprocessing tools may further empower the pPCA technique by facilitating useful visualization functions, such as vessel tracking and automated analysis.

In conclusion, the present disclosure demonstrates that fusing high resolution PCA images with an anatomic MRI data set is an effective and safe perforator imaging technique. The clinical data already demonstrate that this new technique not only has a better safety profile, but also has additional advantages over the current gold-standard of perforator imaging, the CTA, in multiple key features such as image quality, SNR, contrast, and accuracy of perforator anatomy. Thus, the pPCA method is an effective preoperative planning tool for reconstructive surgery using perforator flaps.

What is claimed:

1. A method for Phase Contrast Angiography (pPCA), comprising:
acquiring vascular and flow information using a first MRI sequence;
acquiring anatomic information using a second MRI sequence;
reversing a contrast of the anatomic information to create reversed anatomic information; and
creating a high resolution map of vasculature from the reversed anatomic information and vascular and flow information.

2. The method of claim 1, the first MRI sequence being performed using a phased-array receiver coil having a predetermined number of coil elements to provide a penetration depth within a layer of subcutaneous tissue near the coil surface.

3. The method of claim 2, wherein the phased-array receiver coil provides for visualization of perforator vessels with submillimeter resolution.

4. The method of claim 2, further comprising applying motion control techniques when imaging body parts that are prone to physiologic or voluntary motions.

5. The method of claim 2, wherein the phased-array receiver coil is positioned in close proximity to a body part of interest.

6. The method of claim 1, acquiring the vascular and flow information further comprising using a 3D phase contrast technique.

7. The method of claim 6, wherein a maximum encoded velocity of approximately 15 cm/s is used.

8. The method of claim 6, wherein the vascular and flow information is acquired having a 0.5×0.5 mm in-plane resolution and a 1.5 mm reconstructed slice thickness.

9. The method of claim 1, acquiring the vascular and flow information further comprising using a four-point acquisition scheme to acquire all three orthogonal components of a blood flow velocity vector.

10. The method of claim 1, acquiring anatomic information further comprising post-processing acquired images by inverting a contrast of standard T2-weighted Turbo Spin Echo images to create CTA-like soft-tissue contrast.

11. The method of claim 1, wherein the high resolution map is of an abdominal wall vasculature.

12. The method of claim 1, creating the high resolution map further comprising combining a flow vector field obtained in the vascular and flow information with the reversed anatomic information using image co-registration.

13. The method of claim 1, further comprising displaying the high resolution map,
wherein the high resolution map visualizes a size and location of deep inferior epigastric perforator (DIEP) perforators, and
wherein the high velocity map visualizes the relationship of the DIEP perforators with surrounding tissue and a blood flow velocity within them.

14. The method of claim 13, wherein a product of the blood flow velocity within a perforator vessel and its diameter provides a metric of the perforator vessel's perfusion capability.

15. The method of claim 1, further comprising providing the high resolution map of the vasculature within a computer-assisted surgical planning system.

16. The method of claim 1, further comprising applying the high resolution map of the vasculature to provide personalized flap design in accordance with a patient under study.

17. A magnetic resonance imaging (MRI) apparatus, comprising:
a magnet;
gradient coils;
radio frequency (RF) coils; and
a controller executing instructions to perform a method of Phase Contrast Angiography (pPCA) to:
acquire vascular and flow information using a first MRI sequence;
acquire anatomic information using a second MRI sequence;
reverse a contrast of the anatomic information to create reversed anatomic information; and
create a high resolution map of vasculature from the reversed anatomic information and vascular and flow information.

18. The MRI apparatus of claim 17, wherein the RF coils comprise a phased-array receiver coil, and wherein the first MRI sequence is performed using the phased-array receiver coil having a predetermined number of coil elements to provide a penetration depth within a layer of subcutaneous tissue near the coil surface.

19. The MRI apparatus of claim 18, wherein the phased-array receiver coil is positioned in close proximity to a body part of interest.

20. The MRI apparatus of claim 17, wherein the vascular and flow information is acquired using a four-point acquisition scheme to acquire all three orthogonal components of a blood flow velocity vector.
